(19) United States
(12) Patent Application Publication
(10) Pub. No.: US 2012/0191355 A1
(43) Pub. Date: Jul. 26, 2012

(54) Insect Prediction Techniques for the Forestry Industry

(76) Inventor: Maxine Highsmith, Raleigh, NC (US)

(21) Appl. No.: 12/760,586

(22) Filed: Apr. 15, 2010

(51) Int. Cl. G01N 33/00; G06F 19/00; G09B 23/00

(52) U.S. Cl. 702/19; 434/295

(57) ABSTRACT

A study of the tip moth-pine tree interaction reveals clues as to the moments when the pine trees are most likely to be damaged by tip moths. From this study, software tools may be extrapolated which help farmers know when to apply pesticides to limit damage to the pine crop and also educational software so student-farmers may know more about the life-cycle of the crop and threats to the crop. While originally focused on the pine tree-tip moth dynamic, the study is readily abstracted for use with other crop-insect dynamics.

### STATEMENT OF FEDERAL INTEREST

The invention described herein was made in the performance of work under a USDA-Forest Service contract (Cooperative Research Agreement No. 29-603), and is subject to the provisions of Public Law 96-517 (35 U.S.C. §202) in which the Contractor has elected to retain title.

### FIELD

The present disclosure relates to prediction of insect populations within crops of the forestry industry.

### BACKGROUND

The forestry industry has an interest in growing trees relatively quickly so that the wood may be harvested for myriad uses. Numerous vectors may retard the growth of the trees and reduce the volume of wood available for harvest. Insects that feed on the trees are one such vector. The southeast United States grows large quantities of pine (Pinus sp.) trees as part of its forestry industry. In fact, about fifteen percent of the forestland in the southeast provides greater than fifty percent of the U.S. timber supply. Tip moth (Rhyacionia sp.) damage on young pine seedlings is a serious and longtime problem associated with commercial and natural pine plantations. The loblolly pine is commercially the most important pine tree species, but is also a preferred host of the tip moth insect. Tip moth damage on natural and commercial pine forests may result in a 28-30% reduction in wood volume. As a result, understanding the tip moth is of great concern to the wood, paper, and pulp industries.

While most of the damage to young pine trees in the southeast is caused by Rhyacionia frustrana (order: Lepidoptera, family: Tortricidae, subfamily: Olethreutidae), the Nantucket pine tip moth, it is also true that most natural and commercial pine tree stands across the country are attacked by one or more of the Rhyacionia species of the subfamily Olethreudiae. Research focused on the life cycle and oviposition behavior of the insect suggests that the female tip moth oviposits on the shoots and needles of young pine seedlings. The developing larvae, as they feed and grow, kill the young pine shoot tips. The death of the young pine shoot tips reduces tree growth and productivity, but rarely causes mortality.

While the tip moth is of particular concern to pine forest farmers, other insects and other crops are also of concern. As such, accurate models of insect behavior and life-cycles are of value.

### BRIEF DESCRIPTION OF THE DRAWINGS

FIG. 1 illustrates a plot of tip moth damage versus time based on an experiment run as part of the present disclosure.

FIG. 2 illustrates a second plot of tip moth damage versus time comparing two planting sites based on an experiment run as part of the present disclosure.

FIG. 3 illustrates a third plot of tip moth damage versus time comparing different plots based on an experiment run as part of the present disclosure.

FIG. 4 illustrates a forth plot illustrating a percentage of relative tip moth damage versus time comparing different species.

FIG. 5 illustrates a flow chart setting forth steps of the experiment.

FIG. 6 illustrates a stylized representation of some of the steps of the process of FIG. 5.

FIG. 7 illustrates a flow chart setting forth steps for using software to predict when to apply pesticides.

FIG. 8 illustrates a flow chart setting forth steps for using software in a classroom setting.

### DETAILED DESCRIPTION

The present disclosure provides an empirically derived model of tip moth activity in a plurality of pine stands. From this empirically derived model, estimates may be made by farmers as to when would be an appropriate time to plant, spray pesticides, and harvest trees. Likewise, the model may be embodied in software that may be used as a teaching tool to help farmers and students understand the lifecycle of insects of interest relative to crops of interest. In another embodiment, the model may be used to test relative susceptibility of a new hybrid so as to verify that a new hybrid has a desired genetic characteristic including that of a backcross.

Before the software is explained, the present disclosure provides an overview of how the data was empirically derived for pine trees and tip moths. From this explanation of the experiment, the interested reader may readily ascertain how the experiment could be replicated, with more data points for the same species, or abstracted from the pine/tip moth experiment to other plant/insect species dynamics.

### The Experiment

Twenty-two established homozygous pine parent tree families comprised of four different species produced open-pollinated (OP) seed or were used in controlled-pollinated (CP) crosses that produced interspecific F1 hybrid families, F1-3-way hybrid families, and a testcross family. Open pollinated seed was collected from three of the homozygous parent species; slash (S) pine, shortleaf (Sf) pine, and loblolly (L) pine. The OP and CP seed collection was used in mixed parent-hybrid plantings at two different locations in two different regions of Mississippi. One was located in the Harrison Experiment Forest Station in Saucier, Miss. near Gulfport and the other location was ninety (90) miles north of Saucier in the Chicasawhay Ranger District of the DeSoto National Forest near Ovett, Miss. Each planting included eleven (11) homozygous parent families from OP seed consisting of seven slash (S) pine families, two loblolly (L) pine families, and two shortleaf pine families. There were two interspecific F1-hybrid parents and both had a longleaf (Lf) pine parent. One was Lf crossed to a resistant S pine to form the F1-hybrid (Lf×S) parent and the other one was Lf crossed to a susceptible Sf pine to form the F1-hybrid (Lf×Sf) parent. The remaining eleven homozygous parent tree families were used only in CP crosses and so were not included in the mixed parent-hybrid planting. Families from the CP crosses included three interprovenance [(L×L); (S×S); (Sf×Sf)] families, two interspecific F1-hybrid [(S×L) and (Sf×S)] families, three F1-3-way hybrid [Sf×(Lf×S)] families, and one testcross hybrid [S×(Lf×Sf)] family.

The seeds, which were either produced from CP crosses or were OP seed, were from established orchards in Saucier, Miss. and Rincon, Ga., maintained by the U.S. Forest Southern Institute of Forest Genetics and International Paper (now ArborGen). The one exception was a tree that was established in the Longleaf Tract Experimental Forest in Alexander, La. The seeds were kept at Harrison Experiment Forest Station and those from the loblolly, shortleaf, and slash parent trees were tested to verify that they were homozygous. The seeds were germinated and grown in a Vermiculite-peat (1:1) media placed in Leach tubes and were maintained in a greenhouse at the Southern Institute of Forest Genetics in the Harrison Experimental Forest in Saucier, Miss. The seedlings were maintained in the greenhouse for one year before being outplanted. Specifically, the seedlings were grown in the greenhouse from spring 1998 to spring 1999, and then outplanted and scored for tip moth damage over a natural five-year period.

The study was a randomized complete block design. Each of five blocks at each field site was a non-contiguous plot that mostly contained an eight-tree per family mix of twenty families arranged in rows six feet within and ten feet between that covered approximately 0.22 acres. A few of the controlled pollinated families had less than eight trees per block, but variations in the number of trees per plot were minimal. The fields were site prepared prior to planting, including mowing with a rotary mower (e.g., BUSH HOG®). After plantation establishment, the sites were mown with a rotary mower regularly over the five-year period to reduce weed competition. There were a total of 536 trees at the Saucier, Miss. site near Gulfport and 473 trees at the north site near Ovett, Miss.

In the experiment performed, the tip moth damage was scored twice a year at six-month intervals for the five year span. In contrast to prior models which attempted to evaluate a degree to which the damage had been incurred, the present experiment scores the damage in a binary format. Either the damage is present or absent for each time interval that damage is assessed. The trees were scored in June, when the insect population is known to be relatively high, and again in December, when damage from all four or five of the year's tip moth generations was complete and pupae were overwintering in shoot tips. The tip moth damage was assessed by visual inspection of the whole tree. Adult and pupae tip moth samples were periodically collected and identified as to species. The damage in this case was essentially due to Rhyacionia frustrana: the Nantucket Pine Tip Moth. While the experiment performed evaluated the damage at six-month intervals, this period may be varied without departing from the scope of the present disclosure. Four-month, three-month, two-month, and one-month periods are all specifically contemplated. Still further, periods of every other week, or even weekly, may be used without departing from the scope of the present disclosure.

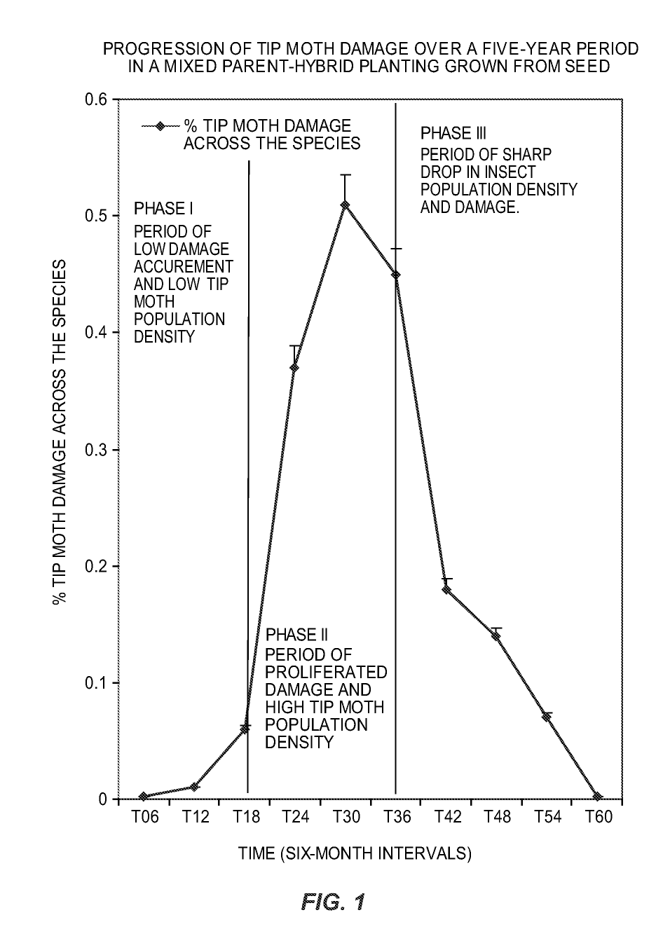

Armed with the data collected as described above, a tip moth population density progression curve may be created as illustrated in FIG. 1. Specifically, the data in FIG. 1 represents the percentages of damage throughout a stand of pine trees at the designated time intervals. As used herein, this measure is referred to as the “percentage of tip moth damage across a stand of trees” or Pₛ. When these percentages of damage are plotted over the entire period of vulnerability in the field, the resulting graph quantitatively depicts the dynamics of the tip moth population density as the insect progressively infests the stand of pine trees. The equation may be defined as follows:

Pₛ=Total # of damaged trees across a pine stand/Grand total number of trees in the planting

Note that the “Grand total number of trees in the planting” should be adjusted for missing or dead trees so the tree count only includes live trees.

From the data of FIG. 1, it is apparent that newly outplanted stands of pine trees are invaded by tip moths during the first year when emergence of the adult tip moth becomes synchronized with growth flushes of the young pine trees. After synchronization, the dynamics of the tip moth population density closely follow changes in the availability of the pine shoot-tips. Three major shifts in the dynamics of the tip moth population density occur as a result of this synchronization, which are denoted in FIG. 1, phase I, phase II, and phase III.

Phase I is characterized by low tip moth density as the tip moth invades the stand of young trees. Not surprisingly, this occurs during the initially slow growth of the young pine trees as they become established and begin development. Tip moth population density increases continuously during phase I as the availability of shoot-tips increases.

Phase II is characterized by a proliferation in the tip moth population density in response to a proliferation in shoot-tip growth during the pine trees' juvenile phase when growth is relatively rapid. The tip moth population density is further increased during phase II in response to adventitious growth of the shoot-tips, which occurs when the tip moth damages the growing tips of the young pine trees. The tip moth population density continues to grow until peak infestation is reached about two and one-half years after stand establishment. A decline in the tip moth population density occurs late in phase II following peak infestation.

The rate of pine tree growth progressively declines over the first three years in the field so that the emergence of the tip moth and growth flushes of the pine trees become increasingly asynchronous until the two become desynchronized late in phase II.

Phase III is marked by the decline in the tip moth population density at the end of phase II and is characterized further by a more drastic decline in the tip moth population density. The pine trees' increase in height is more pronounced during phase III as the tip moth population density is reduced to zero so that the trees no longer accrue tip moth damage.

By design, the experiment and model derived from the experiment smoothes out any variations that may be a result of particular species susceptibility to biological agents, i.e. viruses, chemical factors, i.e. resin flow, climate variations, and soil variations, and to some extent eliminates the need to have in-detail knowledge about the biology of the tip moth. While the biology of the tip moth is not required, it is worth noting that the findings of this experiment are not refuted by prior studies based on insect biology. Rather, the experiment and the previous studies find themselves in remarkable accord. In short, the experiment provides a simple insight into the dynamics of the tip moth population density. Rather than rely on complex pheromone traps to predict insect populations, a review of the population density progression curve shows when the insect populations are high and when it would be appropriate to spray pesticides. A more thorough exploration of this method is set forth below.

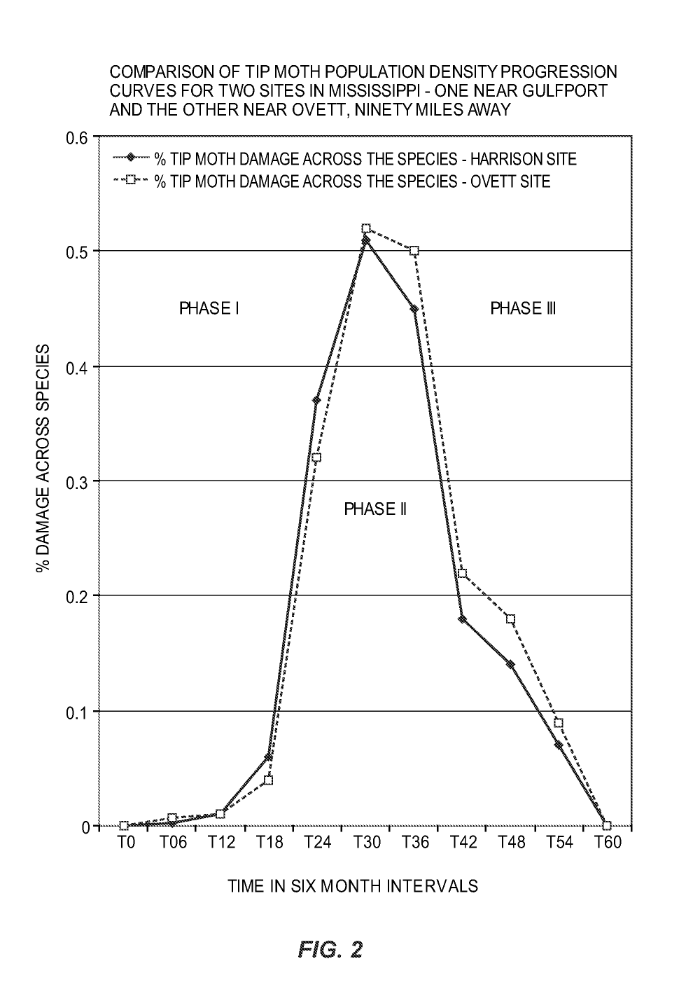

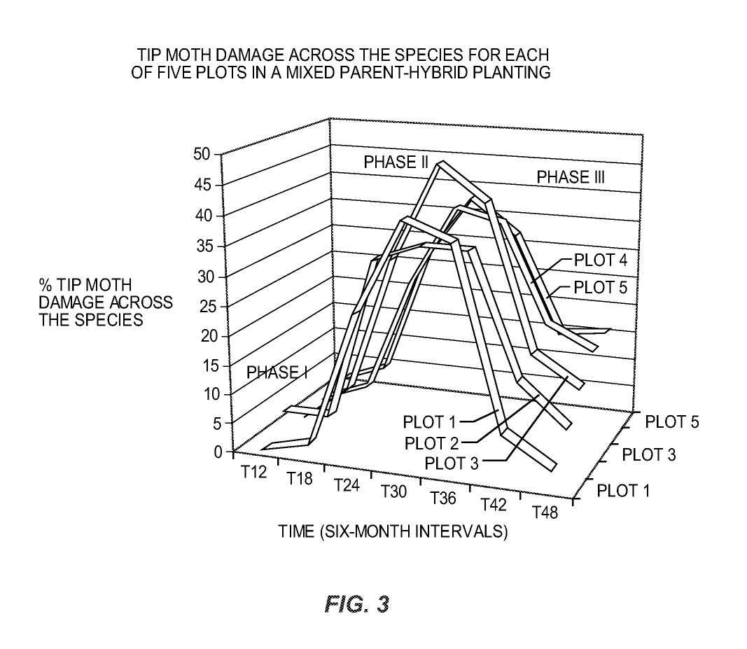

Given that the experiment allows the user to abstract away from particular species specific vulnerabilities, climate changes, and the like, the experiment is readily suitable for use with other species of trees and insects to come up with an insect population density progression curve for that tree-insect dynamic. Armed with the insect population density progression curve, many areas are now available for further development.

Whereas FIG. 1 represents the data extracted from the experiment, FIG. 2 illustrates the differences between data from the two sites used in the experiment. Thus, it is readily apparent how closely the data follows the same trend within the same climatic region. In general, Arkansas, North Carolina, Mississippi, Louisiana, Oklahoma, Texas, Alabama, Georgia, Florida, South Carolina, and Tennessee are considered to be in the same climatic region according to the Köppen Climate Classification for the Conterminous U.S. Thus, the data from this experiment is generally applicable to plantings in these states. FIG. 3 illustrates the same data but broken up by the five plots in a mixed parent-hybrid planting. Again, the three phases are readily apparent across the plantings.

From this experiment, the user is also able to derive a relative susceptibility for each species of pine tree. Based on the relative susceptibility, a pine tree species can be ranked as resistant, intermediately susceptible, or highly susceptible to tip moth damage. The relative susceptibility operates without sophisticated statistical analysis, and thus is amenable to in-field calculations. In particular since the relative susceptibility is based on the dynamics of the tip moth population density and the genetics of the tree species, it requires knowledge only of the number of damaged trees per pine species along with the grand total number of damaged trees from all of the species within the planting.

The relative susceptibility can be calculated for a given tree species within a given planting within a given climatic region for a given time period by multiplying the mean percent of damaged trees for that particular species (“within species”) by 100 and dividing the result by the grand total number of damaged trees throughout the planting (“across the species”) for the same known time interval. The within percent of damaged trees for a particular species is, by convention, calculated by dividing the “within species total number of damaged trees” by the “within species total number of trees” and multiplying the result by 100. The number of trees in all cases should only be the living trees so that missing trees are not included in the count. The equations are as follows.

$$Within Species Mean Percent of Damaged Trees = \frac{within number of damaged trees}{within total number of trees} \times 100$$

$$Relative Susceptibility ( R S ) = \frac{within species mean percent of damaged trees \times 100}{total number of damaged trees from accross the species}$$

RS quantitatively compares the mean percent of damaged trees for a particular tree species within a planting to the total number of damaged trees throughout the planting for a particular time period. This standardizes the comparison of the percentage of damaged trees for the various tree species. The RS of the pine trees is dependent on the genetics of the tree species and the density of the tip moth population with the susceptible tree species becoming more susceptible as the tip moth density increases and the resistant tree species becoming less susceptible. The RS values of the tree species is not changed by the tree sample size.

### Example 1

Suppose that there are 348 trees in a mixed planting consisting of 116 trees each for the three tree species, a loblolly pine parent, a slash pine parent, and their loblolly×slash F1-hybrid pine. The percent of tip moth damage among the species in the mixed planting for the loblolly pine parent, the slash pine parent, and their interspecific loblolly×slash F1 hybrid pine is 90%, 6%, and 92% respectively. The grand total number of damaged trees “across the species” is 218 and the trees were scored during the third year of the planting when tip moth infestation was at its peak. Scoring was based in the knowledge that the susceptibility of the tree species depends both on the genetics of the trees and the density of the tip moth population, with susceptibility being greatest for the susceptible tree species and least for the resistant tree species at peak infestation. The RS values are as follows:

$$R S of loblolly pine = \frac{\frac{90}{100} DAMAGED TREES \times 100}{218 DAMAGED TREES} = 0.41$$

$$R S of slash pine = \frac{\frac{6}{100} DAMAGED TREES \times 100}{218 DAMAGED TREES} = 0.03$$

$$R S of F 1 \text{-} hybrid = \frac{\frac{92}{100} DAMAGED TREES \times 100}{218 DAMAGED TREES} = 0.42$$

Inspection of the RS values in Example I shows a high RS value and a RS value that is significantly lower than an intermediately susceptible value, which would be about one-half of the high RS value. Based on the RS values of the pine tree species in Example 1, the loblolly pine and the interspecific loblolly×slash F1-hybrid have the same high ranking and are classified as susceptible whereas the slash pine species is classified as resistant because its relative ranking is significantly lower than an intermediately susceptible value. While conventional statistics may be applied to the data to determine that the percentages are statistically the same or statistically different, use of the RS values short cuts the process, and makes it more convenient to use RS values with mobile devices having limited computing power.

Susceptibility to tip moth damage (according to the data in this experiment) is density dependent. That is, the greater the density of the tip moth population, the greater the susceptibility of the susceptible species. In other words, the susceptible species become more susceptible as the tip moth population density increases and the resistant species become less susceptible. This phenomenon can be readily seen when the RS of the tree species are calculated across the three phases. Therefore, the susceptible species are most susceptible at peak infestation and the resistant species are least susceptible, as illustrated in Example 1. Peak infestation is the best time to compare the RS of the various tree species. For example the RS of the resistant species will decrease across the three phases while the susceptible species RS will increase from phase I to phase II. The RS of the susceptible species vary in phase III. The RS of loblolly pine is quickly reduced to zero in phase III whereas the RS of shortleaf and its hybrids increase before being reduced to zero.

Since RS values are relative, they may become smaller and approach zero as the tree sample sizes get larger, and the absolute numerical values could change depending on the total number of damaged in the planting but the relative susceptibilities of the tree species will remain the same for an given time period as seen in Example 2.

### Example 2

This example assumes the same species of tree, but with 200 trees in each group. The number of damaged trees changes to 376 and the data is still taken at peak tip moth infestation. The calculations run as follows:

$$R S of loblolly pine = \frac{\frac{90}{100} DAMAGED TREES \times 100}{376 DAMAGED TREES} = 0.24$$

$$R S of slash pine = \frac{\frac{6}{100} DAMAGED TREES \times 100}{376 DAMAGED TREES} = 0.02$$

$$R S of F 1 \text{-} hybrid = \frac{\frac{92}{100} DAMAGED TREES \times 100}{376 DAMAGED TREES} = 0.25$$

The relative susceptibilities of the tree species have not changed and will not change at this tip moth population density, even if the sample size is greatly increased (although the absolute value of the RS values will diminish).

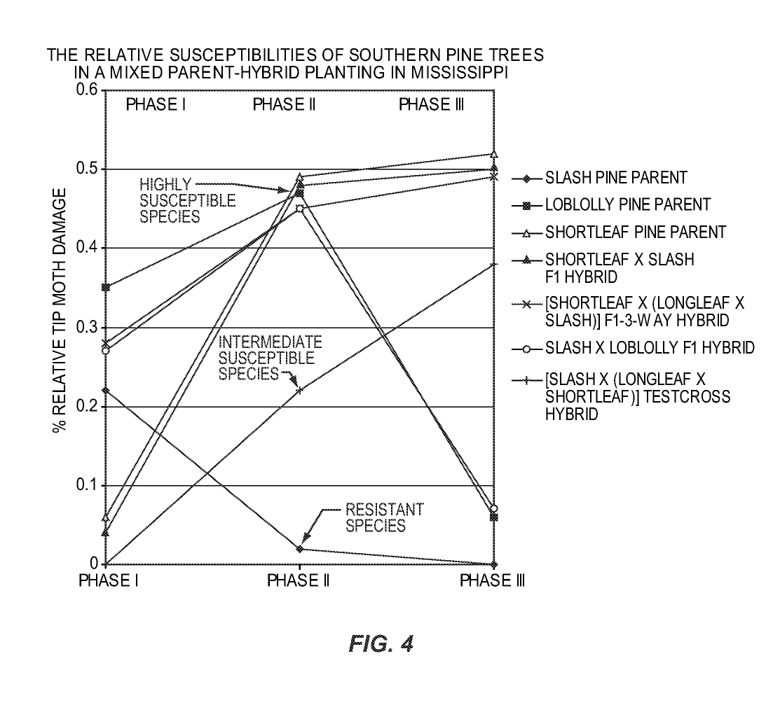

In one exemplary embodiment, the RS may be calculated at different times during growth of the young vulnerable trees and during shifts in the density of the tip moth population. Thus, as illustrated in FIG. 4, a tree that is intermediately susceptible at phase II (i.e., the slash×(shortleaf×longleaf) testcross hybrid) may become more susceptible in phase III, or trees that were highly susceptible in phase II may become less susceptible in phase III (e.g., loblolly and slash×loblolly). Calculating the RS of a tree species across the three phases simply shows how the tree species become more susceptible or resistant as the density of the tip moth population increases or decreases. For example, the resistant species become less susceptible across phases I and II and then go to zero during phase III. The susceptible species become more susceptible across phases I and II as the tip moth density increases to peak infestation and then goes to zero during phase III following the drastic decrease in the tip moth population density that occurs at the end of phase II. Shortleaf, in the Southeast U.S. has often been observed to sustain a relatively greater degree of damage than loblolly and the period of damage for shortleaf has been observed to be extended. The RS values of shortleaf across the phases confirm that the period of time that shortleaf accrue tip moth damage is extended beyond the period of time that loblolly accrue damage. This is also shown to be true for hybrids of shortleaf. Most of the damaged trees late into phase III are due to shortleaf and its hybrids because most of the other trees are no longer accruing damage. Therefore, shortleaf and its hybrids' relative susceptibilities are shown to increase even though their actual genetic propensities for susceptibility have NOT changed.

One of the things that came out of the study is a verification of the results from a previous experiment that suggested that the mechanism of susceptibility of a pine tree species or hybrid to tip moth damage appears to be due to a dominant mode of inheritance. Specifically, the fact that the heterozygous interspecific F1-hybrids were essentially just as susceptible to tip moth damage as their homozygous susceptible parents. A dominant mode of inheritance describes a single gene model in which there is a single major segregating locus with an allele for resistance and an allele for susceptibility, with the susceptible allele being dominant. According to simple Mendelian genetics, when an interspecific, heterozygous susceptible F1-hybrid with one resistant parent and one susceptible parent is crossed back to a homozygous resistant parent, the progeny is heterozygous (segregating) for susceptibility. That is, one-half of the progeny will be resistant and one-half will be susceptible. This progeny is known as a backcross. If the interspecific heterozygous susceptible F1-hybrid is crossed back to a different homozygous resistant parent, the progeny is known as a testcross. However, a testcross and a backcross hybrid are equivalent because they both are a heterozygous F1-hybrid crossed back to a homozygous resistant parent, and the two terms are used interchangeably. Since one-half of a backcross progeny is resistant and one-half is susceptible, this then defines the susceptibility of a backcross pine tree hybrid as one-half of the difference between the damage of the susceptible and resistant parents, which is essentially the same as one-half of the damage of the susceptible parent, whereas the susceptible parent could be a homozygous tree species such as a loblolly pine, or shortleaf pine, or an interspecific (susceptible×resistant) F1-hybrid pine. This is because an interspecific (susceptible×resistant) F1-hybrid is just as susceptible to tip moth damage as its susceptible parent.

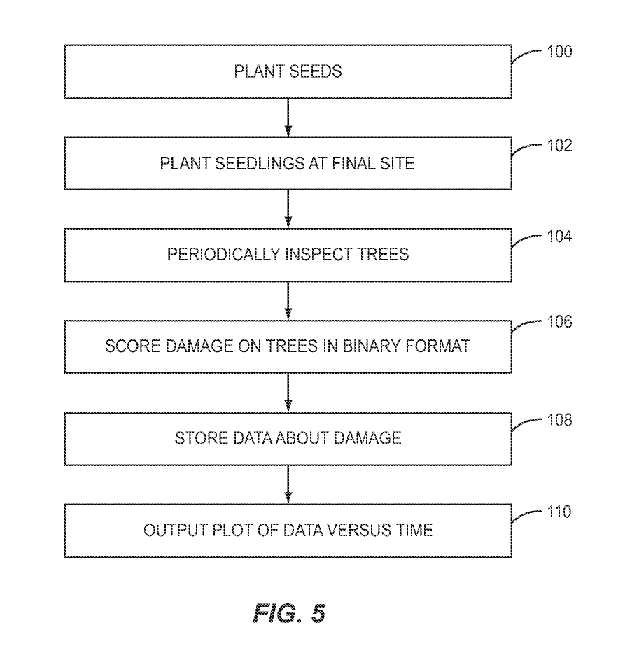

Against this experimental backdrop, several items of interest are worth discussion. The first is the methodology of the experiment. This methodology may be expanded into other plant-insect interactions and is explicated with reference to FIG. 5. Initially, the seeds of interest are cultivated and planted (block 100). This planting may be in a greenhouse, in the field, or other location as desired. If the seeds are initially planted in a greenhouse or other location away from the final planting, then the seedlings are then subsequently transferred to the final plot and planted (e.g., outplanted) (block 102). After planting, the trees are periodically inspected for damage (block 104). While the initial experiment used a six month period between inspections, the period may be shortened to every four months, quarterly (i.e., every three months), every other month, every month, every three weeks, every other week, and even weekly. Manpower constraints suggest that more than once a month is not practical, but would still fall within the scope of the present disclosure.

It is also possible to inspect the trees aperiodically during times of peak interest. For example, the trees could be inspected every month from April to October, and only once otherwise, perhaps in December. That way, the trees are inspected the most when the tip moth are most active and less frequently during the moth's dormant overwintering months. Thus, variations on this aperiodic inspection schedule may be made to accommodate the growth period of the particular climatic region where the trees are planted (e.g., Maine has a shorter growing period, so fewer months of frequent inspection may be used).

During the inspection (regardless of frequency), the damage on the trees is scored in a binary format (block 106). That is, either damage is present or it is absent. The collected data relating to the damage is then stored (block 108). Storage may be on a portable device such as a mobile terminal (e.g., smart phone, laptop) or on paper and then transferred into a computer readable medium (e.g., a database on a computer in a lab or elsewhere). From the data, the computing device may then generate an output of the data versus time (block 110). This output may be on a computer display using software such as MATLAB, output on paper via a printer, or other human readable output as desired.

Note that while trees are particularly contemplated, it is also possible to adapt this process to other sorts of flora as desired. Annuals, perennials, and the like may be used in the process and damage assessed periodically with a corresponding shortening in the periods between inspections.

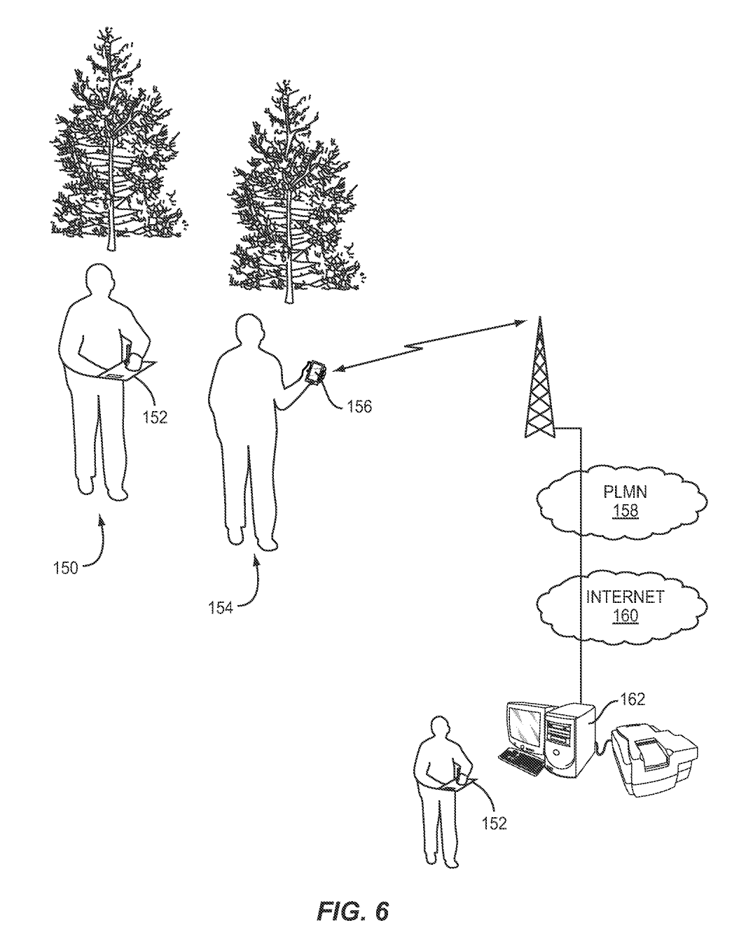

FIG. 6 is a stylized representation of some of the steps in FIG. 5. Specifically at 150, an individual is assessing the trees for damage and recording the data of the assessment using pen and paper 152 (or other writing implements). At 154, an individual is performing the same task using a mobile terminal 156. The mobile terminal 156 may communicate with a computer 162 through the Public Land Mobile Network (PLMN) 158 and/or through the Internet 160 using well known communication protocols. Likewise, the individual using pen and paper 152 may travel to the computer 162 and manually enter the data recorded during the observations. The computer 162 may include a central processing unit (CPU, not shown) and memory with software loaded thereon. Together these act as a control system as that term is defined in the Rules and General Definitions set forth below. The computer 162 also includes a user interface (not labeled) including, but not limited to a mouse, a keyboard, speakers, a monitor/display and a printer. Data may be processed by the control system and output through the user interface including displaying on the monitor and printing on the printer as is well understood.

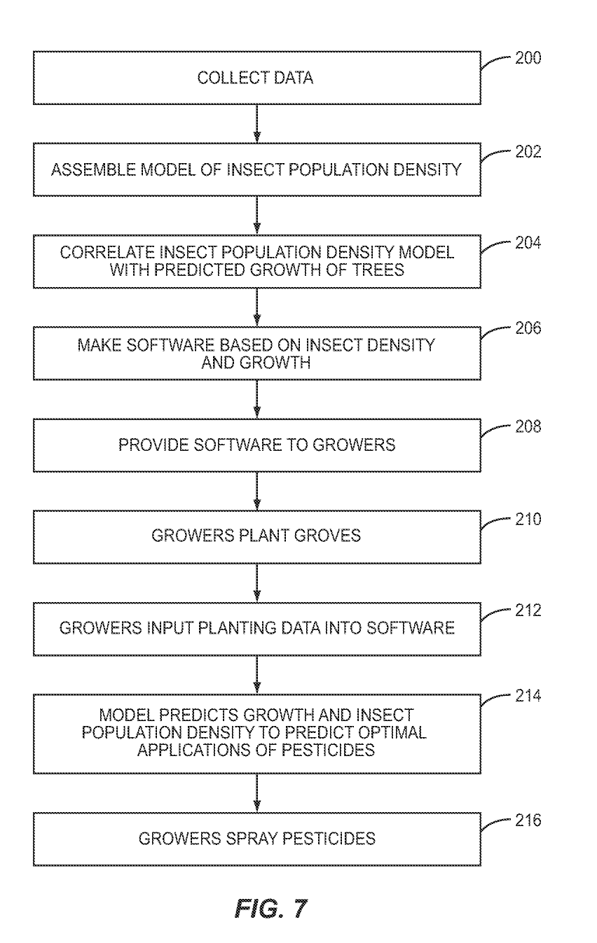

The results of the experiments are also of practical value in that they may help tree growers know when to spray pesticides. An exemplary methodology is set forth with reference to FIG. 7. Initially, the data is collected (block 200) according to the methodology of the experiment set forth above. From this data, an individual may assemble a model of insect population density (block 202). The individual may correlate the insect population density model with a predicted growth of tree model (block 204). The paper at www.ncgia.ucsb.edu/conf/SANTA_FE_CD-ROM/sf_papers/booth_trevor/booth.html describes the challenges and potential solutions to predicting growth of trees and point specifically to C. Hackett's 1991 program PLANTGRO as being one possible mechanism through which growth may be predicted (e.g., Plantgro: a software package for the coarse prediction of plant growth. Melbourne: CSIRO). Using conventional coding techniques, a software package may be created based on the insect population density and the growth models (block 206). The software may include a user interface that accepts hypothetical data regarding when and how many trees were planted as well as geographical data and or climatic data as desired. The software can then use these inputs to predict expected growth and insect population densities using the model as a mechanism to make such predictions.

The software may then be sold or otherwise provided to growers (block 208) and the growers may plant groves of trees (block 210). The growers may input data relating to the planting into the software (block 212). For example, a grower can say that he planted 512 loblolly pine seedlings in a southeastern climate (e.g., Tennessee) on the first of April 2012. The model may then predict the growth of the trees and the insect population density to predict optimal times to apply pesticides (block 214). In an exemplary embodiment, the optimal times to apply pesticides would be immediately prior to peak insect population density. This data may be output to the growers through a user interface of a computer such as by displaying information through a monitor or printing on paper through the printer.

Using the output from the model, the growers then apply the pesticides in an effective amount at the indicated times (block 216) using known pesticide application techniques. In this way, growers may apply the pesticides at the most effective time. As the pesticides are expensive, applying the pesticides at the most effective time may save money for the growers while maximizing harvest. Likewise, this technique avoids the need for elaborate pheromone traps and climate gauges that are currently used to predict when to apply pesticides.

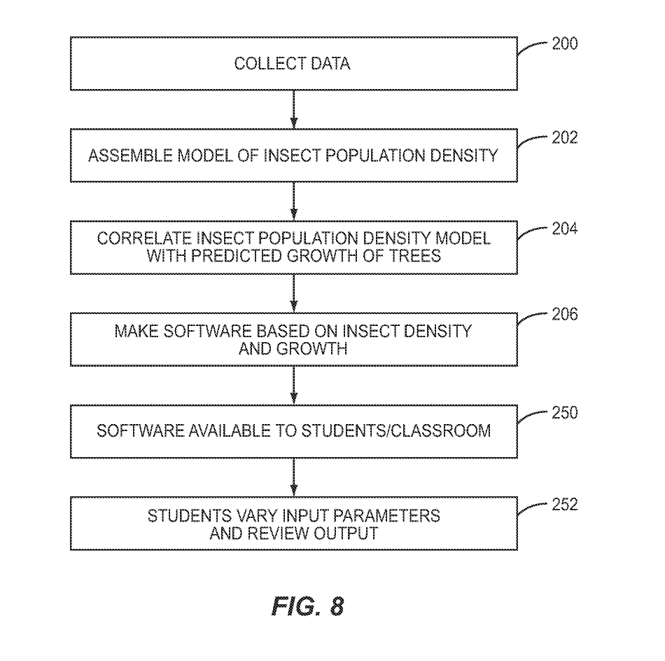

In addition to, or as an alternative to the use of the software by growers, the results of the experiment may also be used as an effective teaching tool for students or interested parties. The process is illustrated with reference to FIG. 8. The process begins in much the same manner in that the data is collected (block 200), an insect population density model is assembled (block 202), correlated with the predicted growth model of the trees (block 204), and software is created (block 206). At this point the methods diverge in that now the software is made available to students or used in a classroom (block 250). In its simplest embodiment, the students may vary input parameters associated with the software to learn how the trees grow and how the population densities of the insect changes over time and/or as a function of one of the input parameters by reviewing any output from the software (block 252). In still another embodiment, the professor may require the students as part of a lab portion of the class to plant a new crop of trees, inspect any previously planted crops for damage and report the data collected so that the model may be iteratively updated.

In still another embodiment, a mobile terminal 156 may be used to calculate relative susceptibilities of trees in the field. For example, an IPHONE™ comes equipped with a calculator and/or an application may be provided on such a mobile terminal which allows simple data entry regarding a number of total trees, a number of damaged trees and species specific information. From this, a control system associated with the mobile terminal 156 may calculate relative susceptibilities.

### RULES OF INTERPRETATION & GENERAL DEFINITIONS

Numerous embodiments are described in this disclosure, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments nor a listing of features of the invention that must be present in all embodiments.

Neither the Title (set forth at the beginning of the first page of this disclosure) nor the Abstract (set forth at the end of this disclosure) is to be taken as limiting in any way as the scope of the disclosed invention(s).

The term “product” means any machine, manufacture and/or composition of matter as contemplated by 35 U.S.C. §101, unless expressly specified otherwise.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

The terms “the invention” and “the present invention” and the like mean “one or more embodiments of the present invention.”

A reference to “another embodiment” in describing an embodiment does not imply that the referenced embodiment is mutually exclusive with another embodiment (e.g., an embodiment described before the referenced embodiment), unless expressly specified otherwise.

The terms “including”, “comprising” and variations thereof mean “including but not limited to”, unless expressly specified otherwise.

The terms “a”, “an” and “the” mean “one or more”, unless expressly specified otherwise.

The term “plurality” means “two or more”, unless expressly specified otherwise.

The term “herein” means “in the present disclosure, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example; the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device or article (whether or not they cooperate) may alternatively be used in place of the single device or article that is described. Accordingly, the functionality that is described as being possessed by a device may alternatively be possessed by more than one device or article (whether or not they cooperate).

Similarly, where more than one device or article is described herein (whether or not they cooperate), a single device or article may alternatively be used in place of the more than one device or article that is described. For example, a plurality of computer-based devices may be substituted with a single computer-based device. Accordingly, the various functionality that is described as being possessed by more than one device or article may alternatively be possessed by a single device or article.

The functionality and/or the features of a single device that is described may be alternatively embodied by one or more other devices that are described but are not explicitly described as having such functionality and/or features. Thus, other embodiments need not include the described device itself, but rather can include the one or more other devices which would, in those other embodiments, have such functionality/features.

Devices that are in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for weeks at a time. In addition, devices that are in communication with each other may communicate directly or indirectly through one or more intermediaries.

A description of an embodiment with several components or features does not imply that all or even any of such components and/or features are required. On the contrary, a variety of optional components are described to illustrate the wide variety of possible embodiments of the present disclosure. Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

Although a process may be described as including a plurality of steps, that does not indicate that all or even any of the steps are essential or required. Various other embodiments within the scope of the described invention(s) include other processes that omit some or all of the described steps. Unless otherwise specified explicitly, no step is essential or required.

Although a product may be described as including a plurality of components, aspects, qualities, characteristics and/or features, that does not indicate that all of the plurality are essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

Headings of sections provided in this disclosure are for convenience only, and are not to be taken as limiting the disclosure in any way.

“Determining” something can be performed in a variety of manners and therefore the term “determining” (and like terms) includes calculating, computing, deriving, looking up (e.g., in a table, database or data structure), ascertaining, recognizing, and the like.

A “display” as that term is used herein is an area that conveys information to a viewer. The information may be dynamic, in which case, an LCD, LED, CRT, LDP, rear projection, front projection, or the like may be used to form the display. The aspect ratio of the display may be 4:3, 16:9, or the like. Furthermore, the resolution of the display may be any appropriate resolution such as 480i, 480p, 720p, 1080i, 1080p or the like. The format of information sent to the display may be any appropriate format such as standard definition (SDTV), enhanced definition (EDTV), high definition (HD), or the like. The information may likewise be static, in which case, painted glass may be used to form the display. Note that static information may be presented on a display capable of displaying dynamic information if desired.

The present disclosure frequently refers to a “control system”. A control system, as that term is used herein, may be a computer processor coupled with an operating system, device drivers, and appropriate programs (collectively “software”) with instructions to provide the functionality described for the control system. The software is stored in an associated memory device (sometimes referred to as a computer readable medium). While it is contemplated that an appropriately programmed general purpose computer or computing device may be used, it is also contemplated that hard-wired circuitry or custom hardware (e.g., an application specific integrated circuit (ASIC)) may be used in place of, or in combination with, software instructions for implementation of the processes of various embodiments. Thus, embodiments are not limited to any specific combination of hardware and software.

A “processor” means any one or more microprocessors, CPU devices, computing devices, microcontrollers, digital signal processors, or like devices. Exemplary processors are the INTEL PENTIUM or AMD ATHLON processors.

The term “computer-readable medium” refers to any medium that participates in providing data (e.g., instructions) that may be read by a computer, a processor or a like device. Such a medium may take many forms, including but not limited to, non-volatile media, volatile media, and transmission media. Non-volatile media include, for example, optical or magnetic disks and other persistent memory. Volatile media include DRAM, which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during RF and IR data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, a USB memory stick, a dongle, any other memory chip or cartridge, a carrier wave, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols. For a more exhaustive list of protocols, the term “network” is defined below and includes many exemplary protocols that are also applicable here.

It will be readily apparent that the various methods and algorithms described herein may be implemented by a control system and/or the instructions of the software may be designed to carry out the processes of the present disclosure.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models, hierarchical electronic file structures, and/or distributed databases) could be used to store and manipulate the data types described herein. Likewise, object methods or behaviors of a database can be used to implement various processes, such as those described herein. In addition, the databases may, in a known manner, be stored locally or remotely from a device that accesses data in such a database. Furthermore, while unified databases may be contemplated, it is also possible that the databases may be distributed and/or duplicated amongst a variety of devices.

As used herein a “network” is an environment wherein one or more computing devices may communicate with one another. Such devices may communicate directly or indirectly, via a wired or wireless medium such as the Internet, Local Area Network (LAN), Wide Area Network (WAN), or Ethernet (or IEEE 802.3), Token Ring, or via any appropriate communications means or combination of communications means. Exemplary protocols include but are not limited to: BLUETOOTH™, TDMA, CDMA, GSM, EDGE, GPRS, WCDMA, AMPS, D-AMPS, IEEE 802.11 (WI-FI), DASH7, IEEE 802.3, TCP/IP, or the like. Note that if video signals or large files are being sent over the network, a broadband network may be used to alleviate delays associated with the transfer of such large files, however, such is not strictly required. Each of the devices is adapted to communicate on such a communication means. Any number and type of machines may be in communication via the network. Where the network is the Internet, communications over the Internet may be through a website maintained by a computer on a remote server or over an online data network including commercial online service providers, bulletin board systems, and the like. In yet other embodiments, the devices may communicate with one another over RF, cellular networks, cable TV, satellite links, and the like. Where appropriate encryption or other security measures such as logins and passwords may be provided to protect proprietary or confidential information.

Communication among computers and devices may be encrypted to insure privacy and prevent fraud in any of a variety of ways well known in the art. Appropriate cryptographic protocols for bolstering system security are described in Schneier, APPLIED CRYPTOGRAPHY, PROTOCOLS, ALGORITHMS, AND SOURCE CODE IN C, John Wiley & Sons, Inc. 2d ed., 1996, which is incorporated by reference in its entirety.

What is claimed is:

1. A method comprising:
periodically measuring damage by an insect to a crop of trees;
plotting a percentage of the insect damage across the crop of trees against time;
examining the plot to determine when peak insect damage occurs for the crop of trees; and
determining when a pesticide should be applied to a future crop of trees based on the peak insect damage.

2. The method of claim 1 wherein periodically measuring damage comprises a binary decision as to the presence or absence of damage.

3. The method of claim 1 wherein periodically measuring damage comprises measuring every six months or every four months.

4. The method of claim 1 further comprising inferring insect population density based on measured insect damage.

5. The method of claim 1 wherein periodically measuring damage comprises recording through a mobile terminal data measured.

6. The method of claim 1 wherein periodically measuring damage comprises recording data on paper and subsequently entering the data on a computing device.

7. A computer readable medium comprising software with instructions to:
receive a measurement of damage by an insect to a crop of trees;
plot a percentage of insect damage across the crop of trees against time;
determine when peak insect damage occurs for the crop of trees;
receive input regarding parameters relating a future crop of trees; and
output in a human readable format information relating to a predicted peak insect population density based on the input parameters.

8. A method comprising:
evaluating damage to trees by insects versus time data;
determining an optimal time to apply a pesticide; and
applying the pesticide to a crop of trees.

9. The method of claim 1 wherein the crop of trees comprises more than one species, the method further comprising:
calculating a first species damage ratio (R_{S1}) by dividing a number of insect damaged trees of a first species (D_{S1}) by a total number of trees for the first species, herein (T_{S1});
calculating a relative susceptibility (RS) to the insect damage for the first species by dividing R_{S1} by the total number of insect damaged trees for the entire crop (D_{T}); and
optionally, calculating the relative susceptibility (RS) to the insect damage for an additional number of species in the crop.

10. A method comprising:
providing software for teaching a student about insect risks to tree crops;
allowing the student to vary one or more parameters within the software, the parameters selected from a group consisting of: tree species, time, and insect type to make selected parameters; and
outputting projected data showing insect damage over time for the selected parameters.

11. The method of claim 1 wherein a human readable output is generated informing when the pesticide should be applied to the future crop of trees.
